(19) United States
(12) Patent Application Publication
(10) Pub. No.: US 2013/0295659 A1
(43) Pub. Date: Nov. 7, 2013

(54) CLOSED TYPE PHOTO-BIO REACTING APPARATUS FOR MICROALGAE

(71) Applicants: Myongji University Industry and Academia Cooperation Foundation, (US); HYUNDAI MOTOR COMPANY, Seoul (KR); KIA MOTORS CORPORATION, Seoul (KR)

(72) Inventors: Dae Young Goh, Busan (KR); Soo Hyun Ha, Seoul (KR); Won Bae Lee, Seoul (KR); Shin Tae Bae, Anyang (KR); Joo Hwan Lim, Incheon (KR); Bum Suk Jung, Yongin (KR); Seul Ki Lee, Cheonan (KR)

(73) Assignees: HYUNDAI MOTOR COMPANY, Seoul (KR); Myongji University Industry and Academia Cooperation Foundation, Yongin (KR); KIA MOTORS CORPORATION, Seoul (KR)

(21) Appl. No.: 13/687,295

(22) Filed: Nov. 28, 2012

(30) Foreign Application Priority Data
May 2, 2012 (KR) 10-2012-0046148

(51) Int. Cl. C12M 1/00

(52) U.S. Cl. CPC C12M 21/02
USPC 435/292.1

(57) ABSTRACT

Disclosed is a closed type photo-bio reacting apparatus for microalgae. The apparatus includes a reactor body, a hollow fiber membrane contact unit, a fluid circulating pump, a light source, and an angle adjusting lift. The reactor body cultures the microalgae. The hollow fiber membrane contact unit is disposed in the reactor body and supplies carbon dioxide to culture solution circulating in the reactor body. The fluid circulating pump circulates the culture solution in the reactor body. The light source irradiates light into the reactor body. The angle adjusting lift adjusts an inclination angle of the reactor body according to an irradiation angle of the light source.

### CROSS-REFERENCE TO RELATED APPLICATION

This application claims under 35 U.S.C. §119(a) the benefit of Korean Patent Application No. 10-2012-0046148 filed May 2, 2012, the entire contents of which are incorporated herein by reference.

### BACKGROUND

(a) Technical Field

The present invention relates to a closed type photo-bio reacting apparatus for microalgae. More particularly, the present invention relates to a closed type photo-bio reacting apparatus for microalgae, which can efficiently supply CO₂ to culture solution and facilitate replacement of a membrane when the membrane is damaged.

(b) Background

With growing concerns on the global environmental issues such as global warming and depletion of fossil fuels, automotive companies have become more environmentally conscientious. One way to limit the effect a vehicle has on the environment, to limit the amount of carbon dioxide that the vehicle emits. One form of biological CO₂ reducing technology involves the fixation of CO₂ by utilizing the photosynthesis of microalgae and another is biodiesel production technology. These two forms of limiting carbon dioxide output are now considered the most realistic alternative for reducing the greenhouse gases because they can be performed at a room temperature and normal pressure and are able to utilize the carbon cycle principle of the natural world.

In particular, in the area of microalgae, the environment in which microalgae needs to in order to rapidly grow needs to be established. There are many factors that effect the growth of microalgae, such as the type reactor, light, temperature, pH, nutrients, and CO₂, all of which need to be optimized.

Generally, microalgae culturing apparatuses are divided into the open pond systems and closed pond systems. In an open pond system, the microalgae are cultured outdoors and in the closed systems a closed reacting apparatus is used. The open pond system uses an open-type water channel or a pond. The initial investment of the open pond system is for the most part reasonable, and its operation is simple, enabling mass-cultivation. However, since the amount of production per unit of volume is quite small and it requires a large installation space in order to be effective. Also, since a reactor for CO₂ fixation needs to be enlarged, a large sum of investment is needed.

In order to overcome the above limitations of the outdoor culture apparatuses, additional studies related to closed systems of various sizes and shapes such as circular or planar are being actively performed. In a closed type reactors, since the system culture solution is isolated from the external atmosphere, gases do not leak out of the reactor, and even though a vent is provided, it is possible to increase the solubility of gas compared to the open pond systems.

In order to supply CO₂ which is essential for the growth of microalgae, a typical CO₂ supply system for utilizing ordinary atmospheric CO₂, or an aeration method for supplying air bubbles from the bottom of the reactor are used. However, the typical CO₂ supply method is often ineffective and expensive. Also, CO₂ supplied after CO₂ is saturated in culture solution is discharged into the atmosphere, making it difficult to know when carbon dioxide has been fixed.

On the other hand, in case of a hollow fiber membrane contact unit that uses a membrane, since CO₂ is supplied by the concentration diffusion through minute pores, CO₂ saturated in culture solution can be measured, and then the concentration can be controlled. In this case, compare to the aeration method, the stress on the microalgae is less, and most of all, the hollow fiber membrane contact unit is suitable for the closed system.

Also, since the hollow fiber membrane contact unit has a greater contact area compared to the aeration method, the area and power necessary for supplying the CO₂ necessary for a large quantity of culture solution can be reduced, and the expansion, replacement and repair are easily implemented by modularization. Also, since the concentration of the CO₂ at the supply side can be easily changed regardless of the flow or concentration of fluids, a desired amount of CO₂ for the culture solution can be supplied through an automation system to maintain a certain concentration level. However, the hollow fiber membrane contact unit also has a few limitations as well.

First, as the amount of microalgae that grows in culture solution increases, microalgae accumulate on the surface or on the angular parts of the hollow fiber membrane, interrupting the supply of CO₂. Also, when a membrane itself or a potting part which fixes the membrane during the culture severely undergoes chemical and physical shocks, a leakage may occur due to perforation and rupture.

In order to solve the above limitations, a new type of hollow fiber membrane contact unit suitable and optimized for a microalgae photo-bio reacting apparatus is needed, and the new type of hollow fiber membrane contact unit needs to be designed so that a membrane can be easily replaced when a fault occurs.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention provides a closed type photo-bio reactor for microalgae that fixes CO₂ in the cells and a closed type photo-bio reacting apparatus for microalgae, which can more quickly supply a desired concentration of CO₂ to culture solution in the reactor compared to a typical aeration method, can structurally overcome biological contamination that may be generated on the surface of the hollow fiber membrane, and can allow the membrane to be easily replaced upon occurrence of breakage or defect of the membrane.

In one aspect, the present invention provides a closed type photo-bio reacting apparatus for microalgae, including: a reactor body configured to culture the microalgae; a hollow fiber membrane contact unit disposed in the reactor body and supplying carbon dioxide to culture solution circulating in the reactor body; a fluid circulating pump configured to circulate the culture solution in the reactor body; a light source irradiating light into the reactor body; and an angle adjusting lift configured to adjust an inclination angle of the reactor body according to an irradiation angle of the light source.

In an exemplary embodiment, the reactor body may be formed using a plurality of cylindrical pipes, and the plurality of cylindrical pipes may be detachably connected to each other via flanges to increase or reduce the volume of the reactor body.

In another exemplary embodiment, the culture solution and the microalgae may flow in the same direction inside the reactor body, and may be supplied with the carbon dioxide necessary for growth of the microalgae due to a concentration difference of a membrane while passing through the hollow fiber membrane contact unit.

The hollow fiber membrane contact unit may include a hollow fiber potting module having a culture solution inlet and a culture solution outlet, and may prevent microalgae from being accumulated on a surface of a membrane while the culture saluting is passing through the hollow fiber membrane potting module. The hollow fiber membrane potting module may be detachably coupled to the hollow fiber membrane contact unit via flanges as well.

In still yet another exemplary embodiment, the hollow fiber membrane contact unit may include a polyvinylidenefluoride (PVDF) hollow fiber membrane that is highly hydrophobic, and the polyvinylidenefluoride hollow fiber membrane may have a pore size of about 0.05 μm to about 0.2 μm and a porosity of about 65% to about 75%.

In a further exemplary embodiment, the light source may include an artificial light source disposed outside the reactor body, and may irradiate light of a wavelength by which the microalgae photosynthesize even in indoor environments.

Other aspects and exemplary embodiments of the invention are discussed infra.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 is a view illustrating a microalgae photo-bio reacting apparatus according to an exemplary embodiment of the present invention;

FIG. 2 is a side view illustrating adjustment of an inclination angle of a reactor body of FIG. 1;

FIG. 3 is a cross-sectional view illustrating a hollow fiber membrane potting module of a hollow fiber membrane contact unit of FIG. 1;

FIG. 4 is a view illustrating an exterior of a hollow fiber membrane contact unit of FIG. 1;

FIG. 5 is a view illustrating an internal structure of the hollow fiber membrane contact unit of FIG. 4; and

FIG. 6 is a graph illustrating increase rates of CO₂ concentration according to an exemplary embodiment of the present invention compared to a typical CO₂ supplying method.

Reference numerals set forth in the Drawings includes reference to the following elements as further discussed below:
10: reactor body
11: cylindrical pipe
12: gas tank
13: culture solution storage tank
14: hollow fiber membrane contact unit
14a: culture solution inlet
14b: culture solution outlet
14c: case
14d: potting module locking unit
15: circulation pump
16: support frame
17: base frame
18: angle adjusting lift
20: hollow fiber membrane potting module
21: acryl pipe
22: hollow fiber membrane
23: epoxy bond
24: support
10: engine room
12: engine
20: cowl lower panel
30: heater blower

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment. In the figures, reference numbers refer to the same or equivalent parts of the present invention throughout the several figures of the drawing.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The above and other features of the invention are discussed infra.

Hereinafter, exemplary embodiments of the present invention will be described in detail with reference to the accompanying drawings.

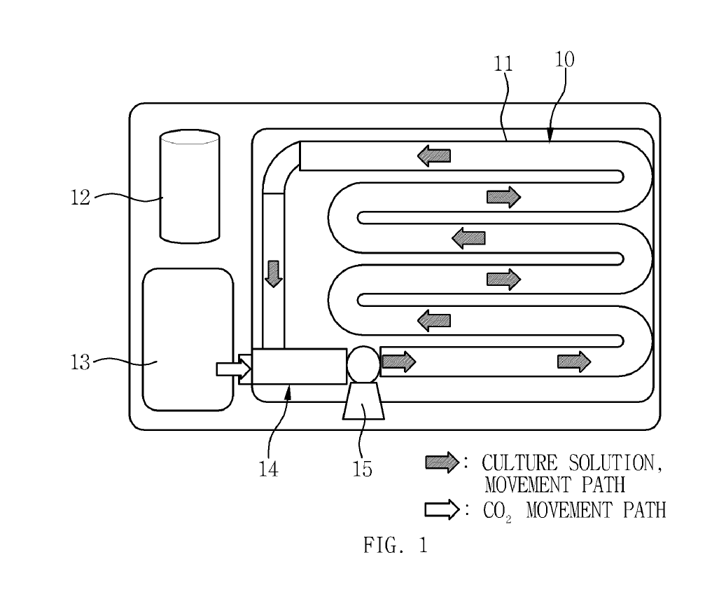

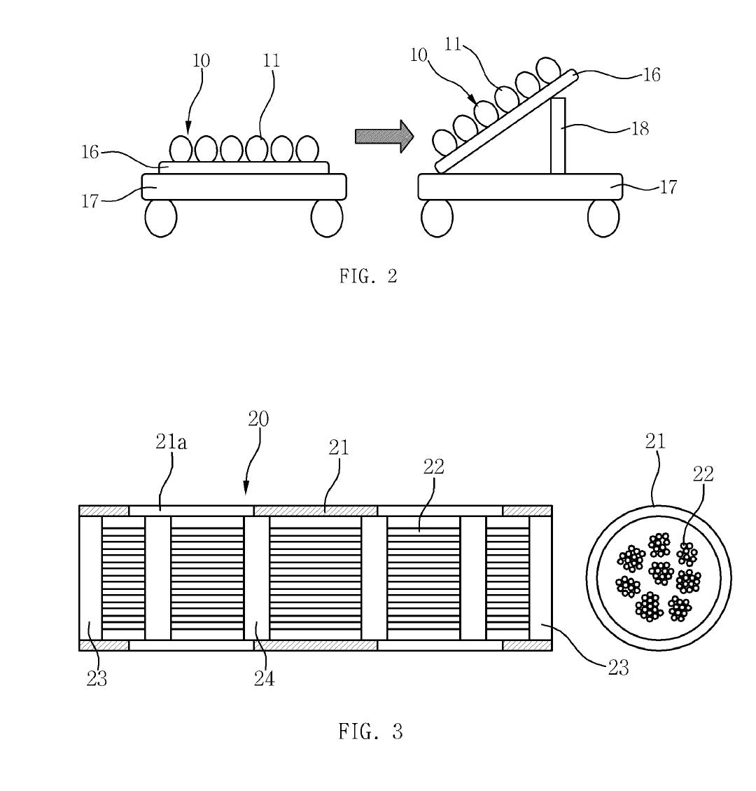

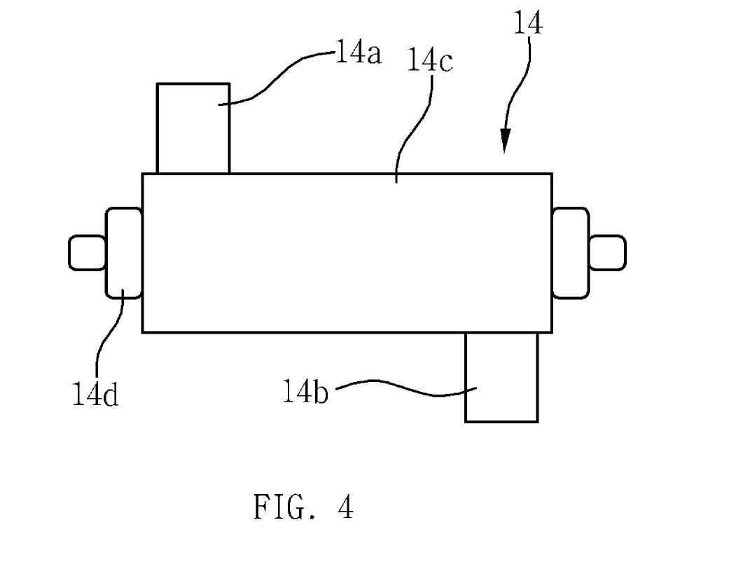

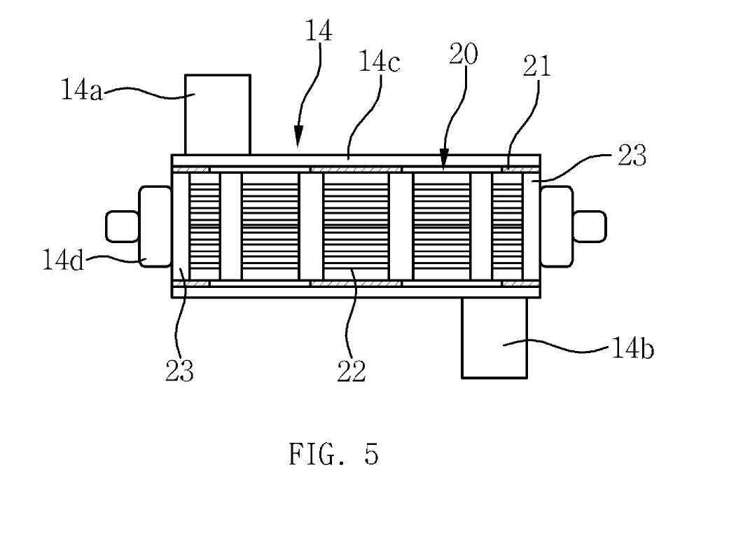

FIG. 1 is a view illustrating a microalgae photo-bio reacting apparatus according to an exemplary embodiment of the present invention. FIG. 2 is a side view illustrating adjustment of an inclination angle of a reactor body of FIG. 1. FIG. 3 is a cross-sectional view illustrating a hollow fiber membrane potting module of a hollow fiber membrane contact unit of FIG. 1. FIG. 4 is a view illustrating the exterior of the hollow fiber membrane contact unit of FIG. 1. FIG. 5 is a view illustrating the internal structure of the hollow fiber membrane contact unit of FIG. 4.

The present invention relates to a closed type photo-bio reacting apparatus for microalgae, which can increase fixation of CO₂ by supplying CO₂ without a separate vent in a closed type reactor using a hollow fiber membrane 22 for mass transfer by concentration diffusion. The present invention can also easily deal with contamination and damage of a membrane by designing a hollow fiber membrane module to be easily mounted and dismounted to/from the reactor.

A microalgae photo-bio reacting apparatus equipped with a hollow fiber membrane contact unit 14 according to an exemplary embodiment of the present invention may include a reactor body 10, a gas tank 12, a microalgae and culture solution storage tank 13, a hollow fiber membrane contact unit 14, a fluid circulating pump 15, and an angle adjusting lift 18. The reactor body 10 may be formed to have a cylindrical shape having a certain volume. The gas tank 12 may store CO₂. The microalgae and culture solution storage tank 13 may store microalgae and culture solution for the adjustment of the initial concentration and the harvest of microalgae. The hollow fiber membrane contact unit 14 may be configured to perform mass transfer and supply CO₂ into a culture solution. The fluid circulating pump 15 may circulate a culture solution. The angle adjusting lift 18 may adjust the angle of the reactor body 10 according to the irradiation angle of a light source. Additionally in some embodiments, the microalgae photo-bio reacting apparatus may include a light supplying apparatus for providing a light source to microalgae. The light source provided to microalgae may be disposed outside the reactor body 10 and may irradiate light having a photosynthesis activating wavelength range into the reactor body 10.

More specifically, the hollow fiber membrane contact unit 14 is shown in detail in FIGS. 3 through 5. FIG. 3 is a side view illustrating a hollow fiber membrane potting module 20 mounted in the hollow fiber membrane contact unit 14 of FIG. 1. As shown in FIG. 3, the hollow fiber membrane potting module 20 may substantially supply CO₂ to a culture solution circulating in a closed type photo-bio reacting apparatus. The hollow fiber membrane potting module 20 may include a plurality of polyvinylidenefluoride (PVDF) hollow fiber membranes 22 for transferring substances between gas and liquid, an epoxy bond 23 for fixing the PVDF hollow fiber membrane 22 and separating gas from the culture solution, an acryl pipe 21 serving as a case of the hollow fiber membrane potting module 22, and a hollow fiber membrane support 24 supporting and protecting the PVDF hollow fiber membrane 22 inside the acryl pipe 21 from a membrane damage caused by the flow rate of the culture solution.

The PVDF hollow fiber membrane 22 may have a tubular structure, a minute diameter and a relatively long length. CO₂ may be transferred to the culture solution through minute pores formed in the membrane. The plurality of PVDF hollow fiber membranes 22 may be disposed as a bundle in the acryl pipe 21 that is a potting module case. CO₂ may be supplied into the respective hollow fiber membranes 22, and the culture solution may be supplied to the external surface of the hollow fiber membrane 22.

The acryl pipe 21 may have a cylindrical shape to surround a bundle of hollow fiber membranes 22. The acryl pipe 21 may have slits at the both sides thereof at a certain interval along the circumferential direction. These slits may be longitudinally formed in the axial direction. The culture solution may be injected into the acryl pipe 21 through the slits (inlet), and then may be discharged from the hollow fiber membrane contact unit 14 to the reactor body through an outlet of the acryl pipe 21. Thereafter, the culture solution may move along the reactor body 10, and then may again flow from the reactor body 10 to the hollow fiber membrane contact unit 14 through the inlet of the acryl pipe 21, thus forming the circulation system of the culture solution.

Also, the epoxy bond 23 may be disposed at the both end portions of the acryl pipe 21. The end portions of the acryl pipe 21 may seal other portions except the end portions of the bundle of hollow fiber membranes 22, supplying CO₂ to both end portions of the hollow fiber membrane 22 and preventing the culture solution from flowing into both end portions of the acryl pipe 21.

Since other portions of both end portions of the acryl pipe 21 except both end portions of the hollow fiber membrane 22 are blocked by the epoxy bond 23, only CO₂ may be supplied through both end portions of the hollow fiber membrane 22, serving to separate gas and liquid.

Due to the above structure of the acryl pipe 21, the culture solution may flow into and out of the acryl pipe 21 through the inlet and the outlet formed on both sides of the acryl pipe 21, and may flow along the outer surface of the hollow fiber membrane 22 accordingly. Also, CO₂ may flow into the hollow fiber membrane 22 by flowing into and out of the both end portions of the hollow fiber membrane 22 exposed to the outside at the both end portions of the acryl pipe 21.

CO₂ flowing in the hollow fiber membrane 22 may be transferred to the culture solution outside the hollow fiber membrane 22 through pores of the hollow fiber membrane 22 by a CO₂ concentration difference. Thus, CO₂ can be supplied to the culture solution. When CO₂ is supplied to the culture solution, microalgae may fix CO₂ through photosynthesis using CO₂ and light supplied from the outside.

FIG. 4 is a detail view of the hollow fiber membrane contact unit 14 mounted in the reactor body 10 of FIG. 1. As shown in FIG. 4, the hollow fiber membrane contact unit 14 may include an inlet 14a and an outlet 14b at both ends of a case 14c thereof, respectively. The inlet 14a and the outlet 14b may induce the culture solution circulating in the reactor body 10 to pass through the hollow fiber membrane contact unit 14. Also, the hollow fiber membrane contact unit 14 may include a gas inlet and a gas outlet for supplying and exhausting a gas mixed with CO₂ together with nitrogen and air, and a potting module locking unit 14d for inserting the hollow fiber membrane potting module 20 shown in FIG. 3 into the hollow fiber membrane contact unit 14 and then fixing the hollow fiber membrane potting module 20 in a flange type.

FIG. 5 is a view illustrating the internal structure of the hollow fiber membrane potting module 20 mounted in the hollow fiber membrane contact unit 14 of FIG. 4. In one embodiment, the reactor body 10 may be a cylindrical polycarbonate (PC) pipe that has a length of about 1.5 m to about 2 m and a diameter of about 10 cm to about 15 cm. The PC pipe may have a light transmittance of about 85%. The PC pipe may be filled with culture solution and microalgae, and all fluids in the pipe may be circulated by the fluid circulating pump 15 in a consistent direction and pass through the hollow fiber membrane contact unit 14. Here, CO₂ necessary for the growth of the microalgae may be supplied through the hollow fiber membrane contact unit 14.

The reactor body 10 may be manufactured using a plurality of cylindrical pipes 11 with flanges at both end portions thereof. The cylindrical pipes 11 may be connected to each other via a connection pipe. Since the flange of the cylindrical pipe 11 and the connection pipe are coupled to each other through screw couplings, the plurality of cylindrical pipes 11 can be easily mounted and dismounted to/from each other in a form of one circulation pipe, and the volume of the reaction body 10 can easily increase or decrease according to a demand of a user. Also since the combination of the cylindrical pipes can be easily dismantled, contaminants or other foreign substances accumulated inside the pipe can be easily removed.

In order words, the plurality of cylindrical pipes (reactor) 11 may be connected to each other via flanges to expand the reactor body 10 according to the capacity of the reactor and necessity. The reactor body may be dismantled to remove contaminants from the inside of the pipe as well.

The microalgae and culture solution storage tank 13 may adjust the initial supply concentration for optimum culture of microalgae and supply nutrients, and may be used for harvest of microalgae. The microalgae and culture solution storage tank 13 may be isolated from the reactor after supplying microalgae and the culture solution to the reactor body 10.

The fluid circulating pump 15 may have a diameter similar to that of the reactor body 10, and may allow culture solution and microalgae inside the reactor to flow at a low rate of about 5 L/min to about 20 L/min. In some embodiments, the fluid circulating pump 15 may include a turbine so that microalgae do not get stressed.

The angle adjusting lift 18 may change the entire height of the reactor body 10 according to the irradiation angle of a light source. For example, the reactor body 10 may be obliquely supported by the angle adjusting lift 18 disposed between a support frame 16 and a base frame 17. The angle adjusting lift 18 may be implemented using hydraulic or pneumatic cylinder and piston, and the inclination angle of the reactor body 10 may be controlled by a method in which the piston moves in the cylinder.

The hollow fiber membrane potting module 20 may be formed using a cylindrical pipe formed of a material such as acryl or polycarbonate. The outer diameter of the pipe may be substantially equal to the inner diameter of the hollow fiber membrane contact unit 14.

The acryl pipe 21 may include an epoxy bond insertion part for fixing the PVDF hollow fiber membrane 22 at both ends thereof, and a hollow part at other portions except a support insertion part for supporting the hollow fiber membrane 22. The acryl pipe 21 may be configured to circulate culture solution through the hollow part, and in this case, may prevent microalgae attachable to the hollow fiber membrane 22 according to the flow of the culture solution from being accumulated in the hollow fiber membrane potting module 20.

The PVDF hollow fiber membrane 22 may serve to supply CO₂ to the culture solution of the reactor body 10. In the hollow fiber membrane control apparatus 14, culture solution containing a low concentration of CO₂ may move along the PVDF hollow fiber membrane 22, the size of minute pores of which may range from about 0.05 μm to about 0.2 μm. The PVDF hollow fiber membrane 22 may be formed of a hydrophobic PVDF material. Accordingly, since the fluid flow pressure is high, and mass transfer between gas and liquid is more efficient than other membrane materials, CO₂ may be more efficiently transferred to the culture solution. Also, since CO₂ is transferred to the culture solution in a gaseous form instead of a liquefied form, CO₂ may be difficult to be again released into the atmosphere, and the transfer speed may be more advantageous than that of a typical aeration type CO₂ supply method.

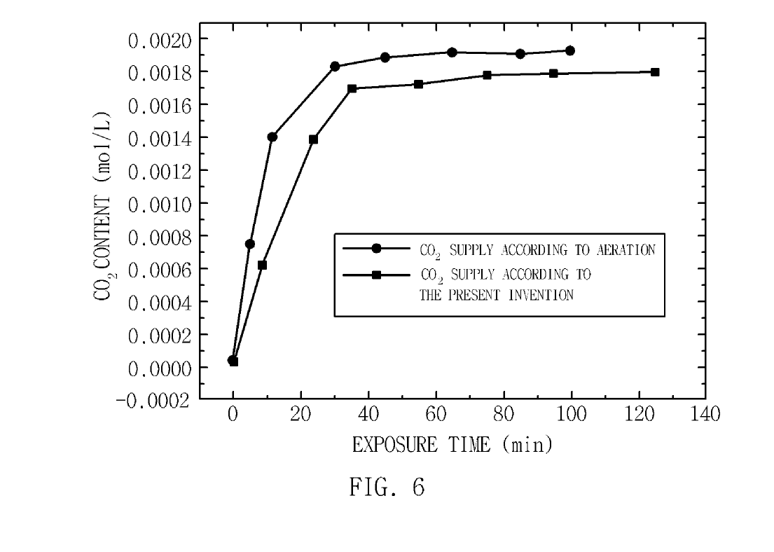

FIG. 6 is a graph showing the increase rate of CO₂ concentration according to a typical CO₂ supplying method. As shown in FIG. 6, the concentration of CO₂ may be increased at a faster speed than aeration in a photo-bio culture medium.

The epoxy bond 23 may fix the hollow fiber membrane 22 at both ends of the acryl pipe 21. In this case, the hollow fiber membrane 22 may be hollow like a straw to allow gas to pass through the hollow fiber membrane 22. According to the operation methods, fluid may flow in the hollow fiber membrane 22, and gas may flow outside the hollow fiber membrane 22.

As shown in FIG. 3, the epoxy bond 23 may fill the inner circumference of both ends of the acryl pipe 21 to prevent the culture solution flowing therein from leaking outside and serve as a fixation method for the hollow fiber membrane 22. Instead of the epoxy bond 23, urethane bond may also be used.

FIG. 4 is a detail view of the hollow fiber membrane contact unit 14, which may be manufactured using a PC pipe or a PVC pipe formed of a cylindrical transparent material. Culture solution may flow into and out of the hollow fiber membrane contact unit 14 through the culture solution inlet 14a and the culture solution outlet 14b. The inlet 14a and the outlet 14b of the hollow fiber membrane contact unit 14 may be configured to correspond to the inlet 14a and the outlet 14b of the hollow fiber membrane potting module 20.

Also, since the potting module locking unit 14d is coupled to both end portions of the hollow fiber membrane contact unit 14 in a flange type by screw coupling after the hollow fiber membrane potting module 20 is inserted into the hollow fiber membrane contact unit 14, the hollow fiber membrane potting module 20 can be easily replaced when a defect occurs in the hollow fiber membrane potting module 20.

A gas inlet and a gas outlet may be disposed over the potting module locking unit 14d to allow gas to flow in and out at both ends of the hollow fiber membrane potting module 20. Therefore, according to exemplary embodiments of the present invention, CO₂ can be saturated in microalgae culture solution at a high speed, and also membrane contamination can be minimized by inserting the hollow fiber membrane potting module 20 into the culture solution circulation type reactor body 10 and allowing all culture solution circulating in the reactor to pass through the hollow fiber membrane 22. Thus, long-term operation can be achieved, and the reactor and the membrane can be easily mounted and dismounted, thereby facilitating the replacement.

A closed type photo-bio reacting apparatus for microalgae according to an embodiment of the present invention has the following advantages.

First, as culture solution passes through a hollow fiber membrane contact unit, CO₂ necessary for the growth of microalgae can be quickly supplied to the culture solution at a desired concentration. Also, a hollow fiber membrane potting module is detachably mounted into the hollow fiber membrane contact unit, therefore the replacement of a membrane can be easily performed when the membrane is damaged.

Second, since the culture solution is allowed to pass through the hollow fiber potting module in the hollow fiber membrane contact unit, contamination of a membrane used in the hollow fiber membrane potting module can be prevented.

Third, since a reactor body can be configured with flanges, the capacity of a reactor can increase or decrease according to necessity, and the fixation rate and efficiency of CO₂ can be improved through microalgae.

The invention has been described in detail with reference to exemplary embodiments thereof. However, it will be appreciated by those skilled in the art that changes may be made in these embodiments without departing from the principles and spirit of the invention, the scope of which is defined in the appended claims and their equivalents.

What is claimed is:

1. A closed type photo-bio reacting apparatus for microalgae comprising:
a reactor body configured for culturing the microalgae;
a hollow fiber membrane contact unit disposed in the reactor body and supplying carbon dioxide to a culture solution circulating in the reactor body;
a fluid circulating pump circulating the culture solution in the reactor body;
a light source irradiating light into the reactor body; and
an angle adjusting lift configured to adjust an inclination angle of the reactor body according to an irradiation angle of the light source.

2. The apparatus of claim 1, wherein the reactor body is formed using a plurality of cylindrical pipes, and the plurality of cylindrical pipes are detachably connected to each other via flanges that are configured to increase or reduce a volume of the reactor body.

3. The apparatus of claim 1, wherein the culture solution and the microalgae flow in the same direction inside the reactor body, and are supplied with carbon dioxide necessary for growth of the microalgae due to a concentration difference of a membrane while passing through the hollow fiber membrane contact unit.

4. The apparatus of claim 1, wherein the hollow fiber membrane contact unit comprises a hollow fiber potting module having a culture solution inlet and a culture solution outlet, and prevents microalgae from being accumulated on a surface of a membrane while the culture solution is passing through the hollow fiber membrane potting module.

5. The apparatus of claim 4, wherein the hollow fiber membrane potting module is detachably coupled to the hollow fiber membrane contact unit via flanges.

6. The apparatus of claim 1, wherein the hollow fiber membrane contact unit comprises a polyvinylidenefluoride (PVDF) hollow fiber membrane that is highly hydrophobic, and the polyvinylidenefluoride hollow fiber membrane has a pore size of about 0.05 μm to about 0.2 μm and a porosity of about 65% to about 75%.

7. The apparatus of claim 1, wherein the light source comprises an artificial light source disposed outside the reactor body, and irradiates light of a wavelength by which the microalgae photosynthesize even at indoor environments.
